(19) United States
(12) Patent Application Publication
(10) Pub. No.: US 2017/0193854 A1
(43) Pub. Date: Jul. 6, 2017

(54) SMART WEARABLE DEVICE AND HEALTH MONITORING METHOD

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Xuchen YUAN, Beijing (CN); Hong ZHU, Beijing (CN); Xin LI, Beijing (CN); Yubing SONG, Beijing (CN); Pengju ZHANG, Beijing (CN)

(21) Appl. No.: 15/251,213

(22) Filed: Aug. 30, 2016

(30) Foreign Application Priority Data
Jan. 5, 2016 (CN) 201610006901.0

(51) Int. Cl. G09B 19/00; A61B 5/053; A61B 5/11; A61B 5/00

(52) U.S. Cl. CPC G09B 19/0092; A61B 5/4872; A61B 5/0537; A61B 5/1118; A61B 5/4866; A61B 5/746; A61B 5/681; A61B 5/6803; A61B 5/1123; A61B 5/742; G09B 19/0038

(57) ABSTRACT

A smart wearable device and a health monitoring method. The smart wearable device includes: an image acquisition unit configured to acquire a first image of food before a wearer takes the food and a second image of the food after the wearer takes the food; a calories analyzing unit configured to analyze the first image and the second image of the food to determine types of the food and an intake amount, and to determine an amount of intake calories based on the types of the food and the intake amount; and a calculation unit configured to generate a target exercise amount based on the amount of intake calories.

### TECHNICAL FIELD

Embodiments of the present disclosure relate to a smart wearable device and a health monitoring method.

### BACKGROUND

Currently, smart bracelets available on the market have functions such as exercise amount monitoring, heart rate monitoring, caller ID display, alarm and sleep quality monitoring, etc. However, as nowadays healthy diet becomes more and more important, these functions of a smart wearable device can hardly meet ever increasing needs from users.

### SUMMARY

Embodiments of the present disclosure provide a smart wearable device. The smart wearable device includes:

an image acquisition unit configured to acquire a first image of food before a wearer takes the food and a second image of the food after the wearer takes the food;

a calories analyzing unit configured to analyze the first image and the second image of the food to determine types of the food and an intake amount, and to determine an amount of intake calories based on the types of the food and the intake amount; and

a calculation unit configured to generate a target exercise amount based on the amount of intake calories.

Embodiments of the present disclosure provide a method of health monitoring used in a smart wearable device, including:

acquiring a first image of food before a wearer takes the food and a second image of the food after the wearer takes the food;

analyzing the first image and the second image of the food to determine types of the food and an intake amount;

determining an amount of intake calories based on the types of the food and the intake amount; and

generating a target exercise amount based on the amount of intake calories.

### BRIEF DESCRIPTION OF THE DRAWINGS

In order to illustrate the technical solutions in the embodiments of the present disclosure or the existing arts more clearly, the drawings need to be used in the description of the embodiments or the existing arts will be briefly described in the following; it is obvious that the drawings described below are only related to some embodiments of the present disclosure, for one ordinary skilled person in the art, other drawings can be obtained according to these drawings without making other inventive work.

FIG. 1 schematically illustrates a smart wearable device in accordance with an embodiment of the disclosure;

FIG. 2 schematically illustrates software and hardware implementation of a smart wearable device in accordance with an embodiment of the disclosure; and

FIG. 3 schematically illustrates a health monitoring method in accordance with an embodiment of the disclosure.

### DETAILED DESCRIPTION

Hereafter, the technical solutions of the embodiments of the present disclosure will be described in a clearly and fully understandable way in connection with the drawings related to the embodiments of the disclosure. It is obvious that the described embodiments are just a part but not all of the embodiments of the present disclosure. Based on embodiments of the present disclosure, all other embodiments obtained by those skilled in the art without making other inventive work should be within the scope of the present disclosure.

Embodiments of the disclosure provide a smart wearable device capable of analyzing diet of a user and recommending an exercise amount accordingly. The device can determine an amount of intake calories of a wearer by analyzing types of food and an amount of food eaten by the wearer. The device then generates a target exercise amount based on the amount of intake calories and presents the target exercise amount to the wearer, such that the wearer may exercise properly to consume an appropriate amount of calories and stay in good health.

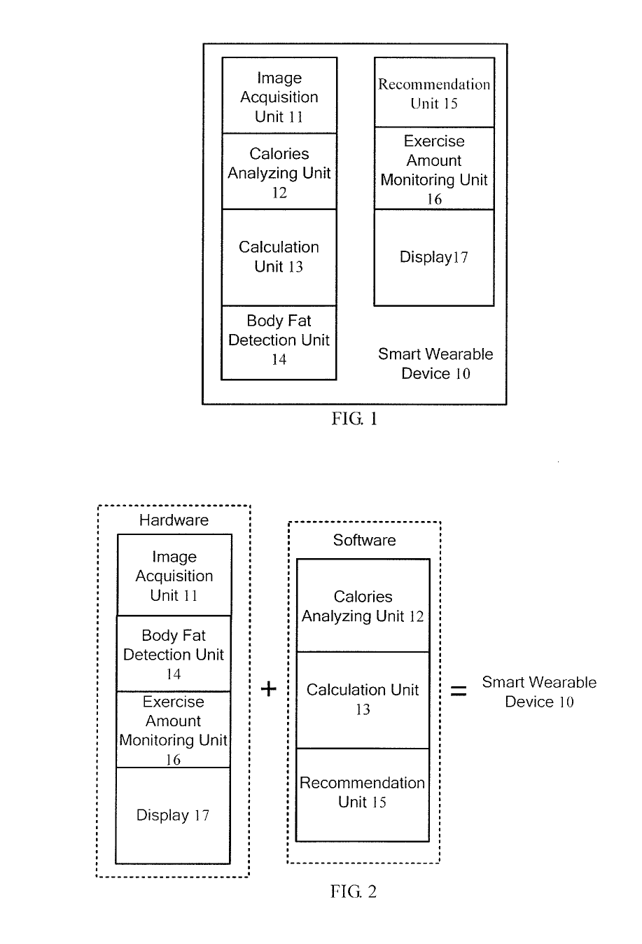

FIG. 1 schematically illustrates a smart wearable device 10 in accordance with an embodiment of the disclosure. The smart wearable device 10 comprises:

an image acquisition unit 11, which is configured to acquire a first image of food before a wearer takes the food and a second image of the food after the wearer takes the food;

a calories analyzing unit 12, which is configured to: analyze the first image and the second image of the food to determine types of the food and an intake amount (e.g., the intake amount is a difference between volumes of the food in the first and second images), and to determine an amount of intake calories based on the types of the food and the intake amount;

a calculation unit 13, which is configured to generate a target exercise amount based on the amount of intake calories.

The device can determine the amount of intake calories of the wearer by analyzing the types and amount of food taken by the wearer. It then generates the target exercise amount based on the amount of intake calories and presents the target exercise amount to the wearer. For example, if a greater amount of intake calories is determined, then a larger target exercise amount is generated; if a smaller amount of intake calories is determined, then a smaller target exercise amount is generated. Thus, the wearer may exercise properly to consume an appropriate amount of calories and stay in good health.

For example, the above device 10 further comprises:

a body fat detection unit 14, which is configured to detect a body fat index of the wearer.

For example, the body fat detection unit 14 detects the body fat index of the wearer by measuring a resistivity of the wearer. Such a unit helps the wearer to determine his or her body fat index in real time. The body fat index can reflect an obesity state of a wearer, which may then be used to accurately determine the wearer's obesity state.

For example, the calculation unit 13 is configured to determine a body shape of the wearer based on the body fat index, a height and a weight of the wearer, and to generate the target exercise amount based on the body shape and the amount of intake calories.

In the embodiment, the height and weight of the wearer may be input by the wearer manually.

For example, in a case that a height of a wearer is larger than a first height (e.g., 185 cm), a weight of the wearer is larger than a first weight (e.g., 90 kg) and a body fat index of the wearer is larger than a predetermined percentage (e.g., 30%), it can be determined that the body shape of the wearer is tall and fat. In a case that a height of a wearer is less than a second height (e.g., 170 cm), a weight of the wearer is larger than a second weight (e.g., 80 kg) and a body fat index of the wearer is larger than a predetermined percentage (e.g.,30%), it can be determined that the body shape of the wearer is short and fat.

For the above two types of wearers that are determined to have an obese body shape (e.g., “tall and fat” or “short and fat”), if the amount of intake calories is bigger than a predetermined amount of calories, then a relatively large target exercise amount may be generated. Moreover, although the first wearer is fat, he/she is taller. In this case, the first wearer may consume more calories than the second wearer when the exercise amount is the same. Even if the two wearers have the same body fat index, a taller wearer will look thinner than a shorter wearer. Thus, a smaller target exercise amount may be generated for the first wearer and a larger target exercise amount may be generated for the second wearer, and so, different target exercise amounts may be generated for different people.

For example, the above device 10 may further comprise:

a recommendation unit 15, which is configured to recommend diet varieties based on the body shape.

The embodiment may help the wearer to choose diet. For example, a low-calories diet may be recommended to the above two obese wearers. Moreover, food rich in calcium may be additionally recommended to a short and thin wearer, which may help the wearer to grow taller.

For example, the recommendation unit may recommend different types of sports and/or different diet varieties based on the types of food. For example, in a case that the wearer has milk, cheese and/or meat, which mainly have saturated fat, regular sports such as jogging and sit-up may be recommended, such that calories generated by the saturated fat may be consumed. As another example, in a case that the wearer has deep fried food such as fried chicken and chips, which mainly have trans fat that is difficult to be consumed by exercising, specific food such as wood-ear and fish may be recommended to the wearer, such that the intake trans-fat may be excreted from the body.

For example, the above device 10 may further comprise:

an exercise amount monitoring unit 16, which is configured to detect an exercise amount of the wearer. For example, the exercise amount monitoring unit 16 may be a pedometer.

For example, the calculation unit 13 is further configured to determine an exercise mode based on the exercise amount and a corresponding time.

For example, when a recorded exercise amount is 5000 steps, and a corresponding time is three hours or above, it can be determined that the exercise mode of the wearer is walking. When the corresponding time is one hour or less, it can be determined that the exercise mode of the wearer is jogging.

For example, the calculation unit 13 is further configured to determine an amount of consumed calories based on the exercise amount, the exercise mode and the body shape of the wearer.

For example, for the same exercise amount, jogging may consume more calories than walking, and a taller and fatter wearer may consume more calories than a shorter wearer under the same exercise amount. Therefore, the consumed calories may be more accurately calculated based on the four parameters above (e.g., an exercise mode, a height and a weight of the wearer, an exercise amount).

For example, the calculation unit 13 is further configured to calculate an amount of increased calories based on the amount of intake calories and the amount of consumed calories. A warning may be given to the wearer if the amount of increased calories is larger than a predetermined calories amount.

The amount of increased calories is approximately equal to the amount of intake calories minus the amount of consumed calories. If the amount of increased calories is larger than a predetermined calories amount (e.g., 3000 calories), body fat of the wearer may probably increase and a warning message may be presented to the wearer.

It is noted that the above parameters and calculation structure may be displayed to the wearer through a display 17 straightforwardly.

The smart wearable device of the embodiments of the disclosure may further comprise one or more processors and one or more memories. The processors may process data signals and may comprise various computing structures, such as Complex Instruction Set Computer (CISC), Reduced Instruction Set Computer (RISC), or a structure implementing a combination of multiple instruction sets. The memories may store instruction and/or data processed by the processors. Such instruction and/or data may comprise codes configured for implementing some or all functions of one or more units in the embodiments of the disclosure. For example, the memories comprise a Dynamic Random Access Memory (DRAM), a Static Random Access Memory (SRAM), a flash memory, an optical memory or other memories well-known by those skilled in the art.

As illustrated in FIG. 2, the image acquisition unit 11, the body fat detection unit 14, the exercise amount monitoring unit 16 and the display 17 may all be implemented using hardware; and the calories analyzing unit 12, the calculation unit 13 and the recommendation unit 15 may be implemented using software.

For example, the image acquisition unit 11 may comprise a camera. The body fat detection unit 14 may comprise a body fat detector. The exercise amount monitoring unit 16 may include a pedometer. The display may comprise a liquid crystal display (LCD) with a touch control function or other types of monitors.

For example, the calories analyzing unit 12, the calculation unit 13 and the recommendation unit 15 may comprise code and programs stored on the memories. The processor may execute the code and programs to implement all or part of the functions of the above calories analyzing unit 12, the calculation unit 13 and the recommendation unit 15.

It can be contemplated that the calories analyzing unit 12, the calculation unit 13 and the recommendation unit 15 may also be implemented using hardware. For example, the calories analyzing unit 12, the calculation unit 13 and the recommendation unit 15 may be specialized hardware devices for implementing all or part of the above functions. For example, the calories analyzing unit 12, the calculation unit 13 and the recommendation unit 15 may be one or more circuits or a combination of multiple circuits for implementing the above functions. In the embodiments of the disclosure, the one or more circuits may comprise: (1) one or more processors; (2) one or more non-transitory computer readable storages connected to a processor; and (3) firmware executable by the processor.

In some embodiments, one or more of the image acquisition unit 11, the body fat detection unit 14, the exercise amount monitoring unit 16, the display 17, the calories analyzing unit 12, the calculation unit 13 and the recommendation unit 15 may be disposed in a smart terminal (such as a smart phone) connected to the smart wearable device. The smart wearable device may exchange data with the smart terminal via wired or wireless links.

For example, the smart wearable device 10 comprises at least one of the following: a smart bracelet, a smart ring and a smart headband.

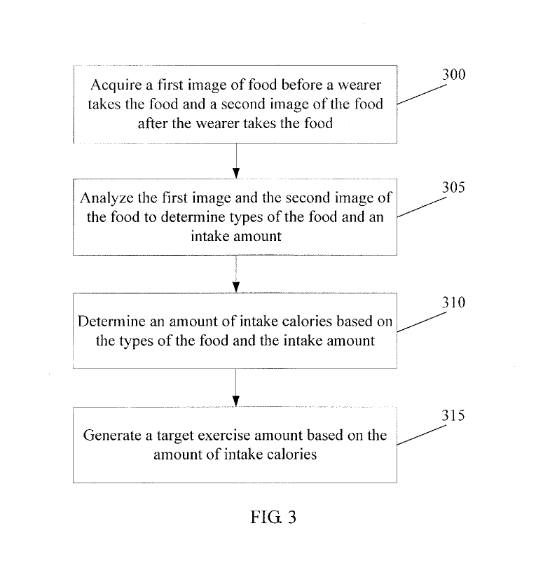

An embodiment of the disclosure further provides a method of health monitoring used in a smart wearable device. As illustrated in FIG. 3, the method comprises:

Step 300: acquiring a first image of food before a wearer takes the food and a second image of the food after the wearer takes the food;

Step 305: analyzing the first image and the second image of the food to determine types of the food and an intake amount;

Step 310: determining an amount of intake calories based on the types of the food and the intake amount; and

Step 315: generating a target exercise amount based on the amount of intake calories.

For example, the method further comprises detecting a body fat index of the wearer.

For example, the step of detecting the body fat index of the wearer comprises detecting the body fat index of the wearer by measuring a resistivity of the wearer.

For example, the step of generating the target exercise amount based on the amount of intake calories comprises: determining a body shape of the wearer based on the body fax index, a height and a weight of the wear; and generating the target exercise amount based on the body shape and the amount of intake calories.

For example, the method further comprises recommending diet varieties based on the body shape.

For example, the method further comprises detecting an exercise amount of the wearer.

For example, the method further comprises determining an exercise mode based on the exercise amount and a corresponding time. A corresponding time may be a time duration when the wearer exercises to achieve the exercise amount.

For example, the method further comprises determining an amount of consumed calories based on the exercise amount, the exercise mode and the body shape of the wearer.

For example, the method further comprises calculating an amount of increased calories based on the amount of intake calories and the amount of consumed calories; and sending a warning message if the amount of increased calories is larger than a predetermined calories amount.

The above description describes the technical solution of the disclosure in details in connection with the drawing. In existing technologies, smart wearable devices cannot analyze a user's diet and recommend exercise amount accordingly. According to the technical solution of the disclosure, it can determine an amount of intake calories of a wearer by analyzing types and amount of food taken by the wearer. It then generates a target exercise amount based on the amount of intake calories and presents the target exercise amount to the wearer, such that the wearer may exercise properly to consume an appropriate amount of calories and stay in good health.

In the present disclosure, terms such as “first”, “second” and the like used in the present disclosure do not indicate any sequence, quantity or significance but only for distinguishing different constituent parts. Also, the terms such as “a,” “an,” or “the” etc., are not intended to limit the amount, but indicate the existence of at lease one. The terms “comprises,” “comprising,” “includes,” “including,” etc., are intended to specify that the elements or the objects stated before these terms encompass the elements or the objects and equivalents thereof listed after these terms, but do not preclude the other elements or objects.

The foregoing are merely specific embodiments of the disclosure, but not limitative to the protection scope of the disclosure. One skilled in the art could devise variations or replacements that within the scope and the spirit of the present disclosure, those variations or replacements shall belong to the protection scope of the disclosure. Thus, the protection scope of the disclosure shall be defined by the accompanying claims.

The present disclosure claims the benefits of Chinese patent application No. 201610006901.0, which was filed on Jan. 5, 2016 and is incorporated herein in its entirety by reference as part of this application.

What is claimed is:

1. A smart wearable device, comprising:
an image acquisition unit configured to acquire a first image of food before a wearer takes the food and a second image of the food after the wearer takes the food;
a calories analyzing unit configured to:
analyze the first image and the second image of the food to determine types of the food and an intake amount; and
determine an amount of intake calories based on the types of the food and the intake amount; and
a calculation unit configured to generate a target exercise amount based on the amount of intake calories.

2. The device of claim 1, further comprising:
a body fat detection unit configured to detect a body fat index of the wearer.

3. The device of claim 2, wherein the body fat detection unit detects the body fat index of the wearer by measuring a resistivity of the wearer.

4. The device of claim 2, wherein the calculation unit is configured to determine a body shape of the wearer based on the body fax index, a height and a weight of the wear, and to generate the target exercise amount based on the body shape and the amount of intake calories.

5. The device of claim 4, further comprising:
a recommendation unit configured to recommend diet varieties based on the body shape.

6. The device of claim 4, further comprising:
an exercise amount monitoring unit configured to detect an exercise amount of the wearer.

7. The device claim 6, wherein the calculation unit is further configured to determine an exercise mode based on the exercise amount and a corresponding time.

8. The device of claim 7, wherein the calculation unit is further configured to determine an amount of consumed calories based on the exercise amount, the exercise mode and the body shape of the wearer.

9. The device of claim 8, wherein the calculation unit is further configured to calculate an amount of increased calories based on the amount of intake calories and the amount of consumed calories, and to present a warning message if the amount of increased calories is larger than a predetermined calories amount.

10. The device of claim 1, wherein the device comprises at least one of a smart bracelet, a smart ring and a smart headband.

11. A method of health monitoring used in a smart wearable device, comprising:
acquiring a first image of food before a wearer takes the food and a second image of the food after the wearer takes the food;
analyzing the first image and the second image of the food to determine types of the food and an intake amount;
determining an amount of intake calories based on the types of the food and the intake amount; and
generating a target exercise amount based on the amount of intake calories.

12. The method of claim 11, further comprising:
detecting a body fat index of the wearer.

13. The method of claim 12, wherein detecting the body fat index of the wearer comprises detecting the body fat index of the wearer by measuring a resistivity of the wearer.

14. The method of claim 12, wherein generating the target exercise amount based on the amount of intake calories comprises:
determining a body shape of the wearer based on the body fax index, a height and a weight of the wearer; and
generating the target exercise amount based on the body shape and the amount of intake calories.

15. The method of claim 14, further comprising:
recommending diet varieties based on the body shape.

16. The method of claim 14, further comprising:
detecting an exercise amount of the wearer.

17. The method of claim 16, further comprising:
determining an exercise mode based on the exercise amount and a corresponding time.

18. The method of claim 17, further comprising:
determining an amount of consumed calories based on the exercise amount, the exercise mode and the body shape of the wearer.

19. The method of claim 18, further comprising:
calculating an amount of increased calories based on the amount of intake calories and the amount of consumed calories; and
presenting a warning message if the increased calories is larger than a predetermined calories amount.
